(19) United States
(12) Patent Application Publication
(10) Pub. No.: US 2026/0047814 A1
(43) Pub. Date: Feb. 19, 2026

(54) SYSTEMS, DEVICES AND METHODS RELATED TO PERIODONTAL MEASUREMENTS WITH RADIOCONTRAST AGENTS

(71) Applicants: Stephanie KWON, Quincy, WA (US); Paul KWON, Quincy, WA (US)

(72) Inventors: Stephanie KWON, Quincy, WA (US); Paul KWON, Quincy, WA (US)

(21) Appl. No.: 19/302,380

(22) Filed: Aug. 18, 2025

Related U.S. Application Data
(60) Provisional application No. 63/684,421, filed on Aug. 18, 2024.

(51) Int. Cl. A61B 6/00; A61B 6/03; A61B 6/40; A61B 6/51; G06T 7/00; G06T 7/62

(52) U.S. Cl. CPC A61B 6/481; A61B 6/03; A61B 6/4085; A61B 6/512; G06T 7/0012; G06T 7/62; G06T 2207/30036

(57) ABSTRACT

A method for measuring a periodontal condition can include providing a radiocontrast agent to a region about a tooth and a respective gum tissue, such that some of the radiocontrast agent enters a gum pocket between the tooth and the gum tissue. The method can further include obtaining radiograph data representative of an image that includes a pocket image resulting from the presence of the radiocontrast agent within the gum pocket, and determining a depth dimension of the gum pocket from the pocket image.

### CROSS-REFERENCE TO RELATED APPLICATION(S)

This application claims priority to U.S. Provisional Application No. 63/684,421 filed Aug. 18, 2024, entitled SYSTEMS, DEVICES AND METHODS RELATED TO PERIODONTAL MEASUREMENTS WITH RADIOCONTRAST AGENTS, the disclosure of which is hereby expressly incorporated by reference herein in its entirety.

### BACKGROUND

### Field

The present disclosure relates to systems, devices and methods for periodontal measurements with radiocontrast agents.

### Description of the Related Art

Periodontal disease typically involves infection and inflammation of gum and bone associated with a given tooth. When in a more advanced form, such a periodontal disease can include the gum separating from the tooth and/or loss of bone supporting the tooth.

In some dental applications, periodontal disease can be detected and/or characterized by, for example, measurement of depth of a pocket formed between a given tooth and the gum separating from the tooth. Such a pocket-depth measurement is typically achieved utilizing a mechanical probe device.

### SUMMARY

In some implementations, the present disclosure relates to a method for measuring a periodontal condition. The method includes providing a radiocontrast agent to a region about a tooth and a respective gum tissue, such that some of the radiocontrast agent enters a gum pocket between the tooth and the gum tissue. The method further includes obtaining radiograph data representative of an image that includes a pocket image resulting from the presence of the radiocontrast agent within the gum pocket, and determining a depth dimension of the gum pocket from the pocket image.

In some implementations, the present disclosure relates to a periodontal device that includes a tray for a patient. The tray is configured to provide a radiocontrast agent to a region about a tooth of the patient and a respective gum tissue, such that some of the radiocontrast agent enters a gum pocket between the tooth and the gum tissue when the tray is worn by the patient. A radiograph image of the tooth and the gum tissue provides a pocket image resulting from the presence of the radiocontrast agent within the gum pocket thereby allowing determination of a depth dimension of the gum pocket from the pocket image.

In some implementations, the present disclosure relates to a system for measuring a periodontal condition. The system includes a radiocontrast application component configured to deliver a radiocontrast agent to a region about a tooth and a respective gum tissue, such that some of the radiocontrast agent enters a gum pocket between the tooth and the gum tissue. The system further includes a radiograph component configured to obtain radiograph data representative of an image that includes a pocket image resulting from the presence of the radiocontrast agent within the gum pocket. The system further includes a computing component configured to determine a depth dimension of the gum pocket from the pocket image.

In some implementations, the present disclosure relates to a periodontal kit that includes a packaging suitable for periodontal application, and a tray for a patient.

The tray is configured to provide a radiocontrast agent to a region about a tooth of the patient and a respective gum tissue, such that some of the radiocontrast agent enters a gum pocket between the tooth and the gum tissue when the tray is worn by the patient, and such that a radiograph image of the tooth and the gum tissue includes a pocket image resulting from the presence of the radiocontrast agent within the gum pocket thereby allowing determination of a depth dimension of the gum pocket from the pocket image. The kit further includes an instruction for use of the tray.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1 shows a sectional view of a periodontal device positioned over a tooth and portions of gum tissues.

FIG. 2 shows a sectional view of the periodontal device of FIG. 1 in use, where a space is occupied with a fluid.

FIG. 3 depicts a computed tomography (CT) image representative of the in-use example of FIG. 2.

FIG. 4 shows an enlarged view of a portion of FIG. 3.

FIG. 5 depicts an image that includes a CT image similar to the CT image of FIG. 3 combined with a contour obtained from surface image data.

FIG. 6 shows an enlarged view of a portion of FIG. 5.

FIG. 7 show that in some embodiments, ambiguity can be removed or reduced during an analysis of a CT image such as the combined-data image of FIG. 5.

FIG. 8A shows a sectional view of a periodontal device with a space occupied with a fluid similar to the example of FIG. 2.

FIG. 8B depicts a CT image representative of the in-use example of FIG. 8A.

FIG. 9 shows that in some embodiments, a periodontal device having one or more features as described herein can be part of a kit.

### DETAILED DESCRIPTION OF SOME EMBODIMENTS

The headings provided herein, if any, are for convenience only and do not necessarily affect the scope or meaning of the claimed invention.

Disclosed are examples related to systems, methods and devices for measurement of one or more periodontal conditions such as gum tissue pocket depth. Although various examples are described herein in the context of gum tissue pocket depth measurements, it will be understood that one or more features of the present disclosure can also be utilized to measure other periodontal conditions.

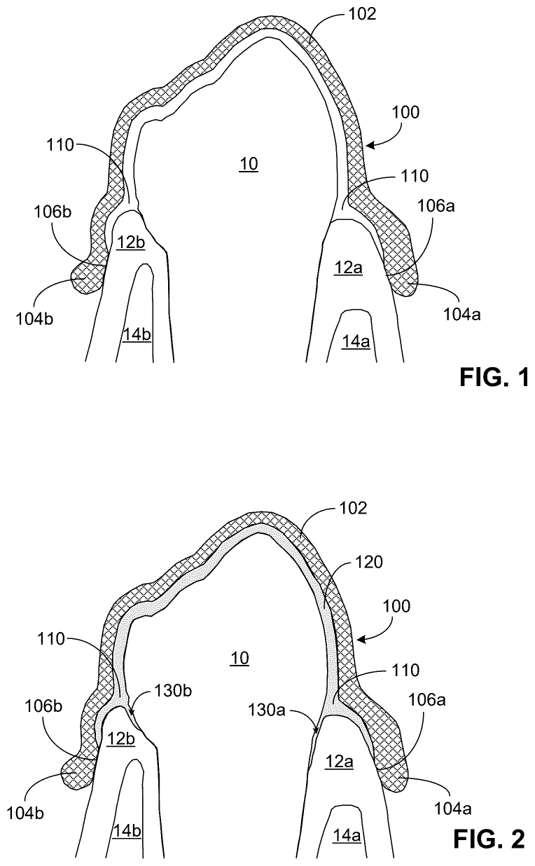

FIG. 1 shows a sectional view of a periodontal device 100 positioned over a tooth 10 and portions of gum tissues 12a, 12b. For the purpose of description, the gum tissue 12a can be on the facial/buccal side, and the gum tissue 12b can be on the lingual side of the example tooth 10. Also for the purpose of description, it will be understood that the periodontal device 100 can be configured to cover some or all of the teeth in an upper or lower arch. For example, the periodontal device 100 of FIG. 1 can be configured to cover all of the teeth in an upper or lower arch with appropriate sealing functionality at the facial-side gum tissue, lingual-side gum tissue, and gum tissues behind the rear-most molars.

FIG. 1 shows that in some embodiments, the periodontal device 100 can include a tooth-contour portion 102 having an inner surface that is based on a contour of the tooth 10 to provide a space 110 therebetween. For example, the inner surface of the periodontal device 100 can be configured to follow the contour of the tooth 10, such that the space 110 between the inner surface of the periodontal device 100 and the surface of the tooth 10 is sufficiently spacious to accommodate a fluid around the tooth 10 as described herein.

In some embodiments, the periodontal device 100 can include an outer surface that is based on a contour of the tooth 10 to thereby minimize or reduce the level of discomfort when worn by a user.

FIG. 1 shows that in some embodiments, the periodontal device 100 can be configured such that the space 110 it provides extends to a location beyond a gumline on the respective side of the tooth 10. Thus, on the facial side of the tooth 10, the space 110 is shown to extend beyond the gumline of the gum tissue 12a; and similarly, on the lingual side of the tooth 10, the space 110 is shown to extend beyond the gumline of the gum tissue 12b.

FIG. 1 shows that in some embodiments, the periodontal device 100 can be configured to include a sealing portion at or near its ends 104a, 104b (when viewed in a cross-sectional manner). For example, the ends 104a, 104b can be dimensioned to allow positioning of the periodontal device 100 over the tooth and provide sealing engagements 106a, 106b, respectively, such that the space 110 is sealed sufficiently to allow use of a fluid captured therein. Examples of use of such a fluid are described herein in greater detail.

FIG. 2 shows a sectional view of the periodontal device 100 of FIG. 1 in use, where the space 110 is occupied with a fluid 120. In some embodiments, such a fluid can include a compound that provides image enhancing functionality. For example, the fluid 120 can include a radiocontrast agent such as iodine-based compound or barium sulfate compound for x-ray based imaging such as computed tomography (CT) (e.g., cone beam computed tomography (CBCT)). For the purpose of description, it will be understood that a fluid can be, for example, a liquid, a solution, a suspension, a slurry, or any material capable of being captured in a confined space and flow therein.

In the example of FIG. 2, the fluid 120 is shown to have worked into gum pockets 130a, 130b associated with the respective gum tissues 12a, 12b. Accordingly, a CT image such as a CBCT image can include a contrast-enhanced structure representative of the respective gum pocket, and thereby allow one or more measurements of the gum pocket.

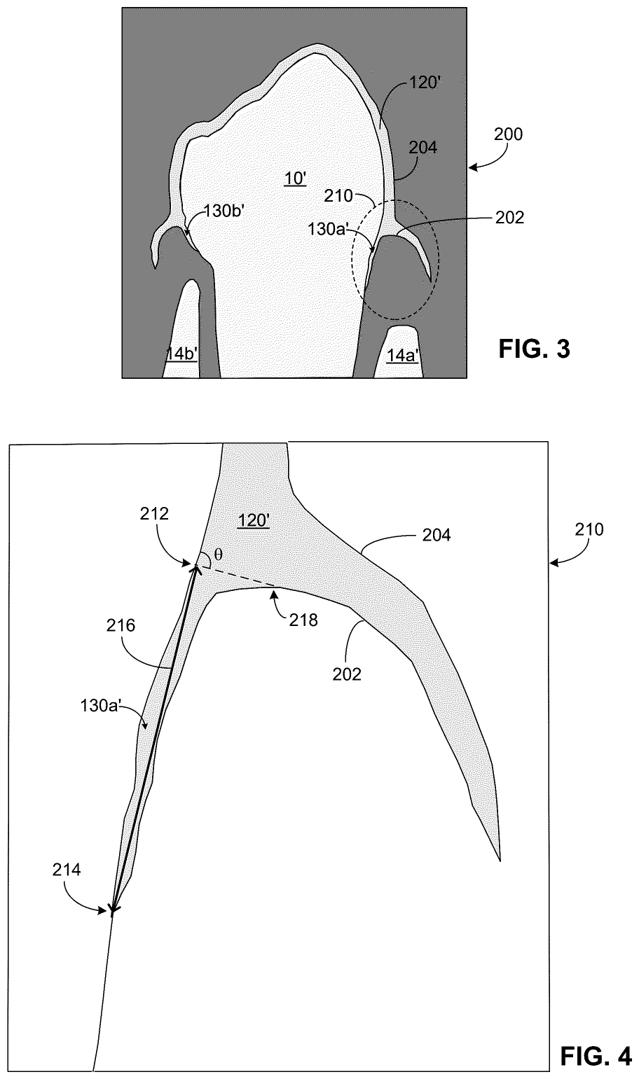

FIG. 3 depicts a CT image 200 representative of the in-use example of FIG. 2, and an enlarged view of an area indicated as 210 is shown in FIG. 4. In such a CT image, the gum tissues (12a, 12b in FIG. 2) are typically not detectable. However, and as shown in FIGS. 2 and 3, contrast-enhanced area 120′ resulting from the presence of the fluid (120 in FIG. 2) includes a boundary 204 provided by the inner surface of the periodontal device (100 in FIG. 2) and a boundary 202 of the corresponding gum tissue. Such a boundary of the gum tissue is shown to include the gum-side portion of the contrast-enhanced structure representative of the respective gum pocket (130a′ in FIG. 4, and also referred to herein as a gum pocket image).

Referring to FIG. 4, the gum pocket image 130a′ is shown to include an open end 212 and a base end 214. The base end 214 is shown to be approximately at a location where the tip of a periodontal probe can be positioned during a probing procedure. Thus, a length measurement (depicted as length of an arrow 216) between the open end 212 and the base end 214 of the gum pocket image 130a′ can provide a gum pocket measurement without a physical probing procedure.

FIG. 4 also shows an example of how the open end 212 of the gum pocket image 130a′ can be estimated. In such an example, the open end 212 can be at a location where a linear line from a peak location 218 of the gum tissue boundary 202 intersects with a tangent line at the open end 212 by an angle θ (e.g., 90 degrees).

In the example of FIG. 4, it is noted that the location of the open end 212 is based on a projection from the peak location 218 of the gum tissue boundary 202. In some situations, such a gum tissue boundary may vary due to, for example, variation in gum tissue profile itself and/or variation in pliability of gum tissue when under pressure from the presence of the fluid (120 in FIG. 2). Such variability in the gum tissue boundary may or may not affect the measured pocket measurement 216 in a significant manner.

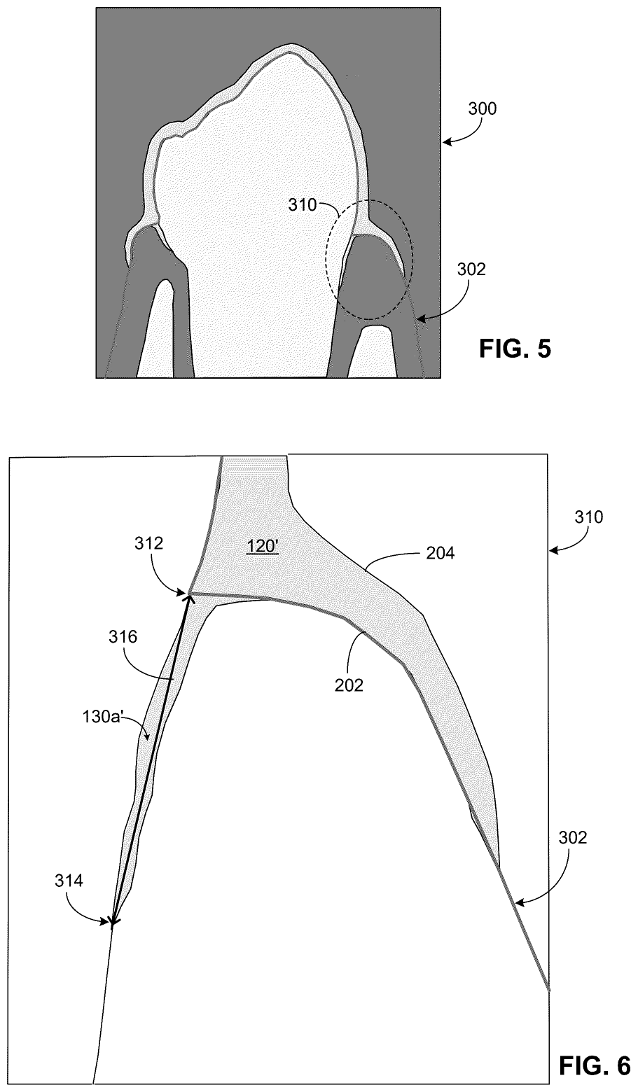

In some embodiments, a location of an open end of a gum pocket image as described herein can be obtained from combined data that includes CT image and surface image data. For example, FIG. 5 depicts an image 300 that includes a CT image (e.g., a CBCT image) similar to the CT image 200 of FIG. 3 combined with a contour 302 obtained from surface image data (e.g., intraoral scan data), and an enlarged view of an area indicated as 310 is shown in FIG. 6.

Additional details related to how CT image and surface image data can be combined and utilized are provided in PCT Publication No. WO 2023/097045 which is expressly incorporated by reference in its entirely, and its disclosure is to be considered part of the specification of the present application.

Referring to the example of FIG. 6, in such an image representative of the combined data, contrast-enhanced area 120′ resulting from the presence of the fluid (120 in FIG. 2) includes a boundary 202 of the corresponding gum tissue, similar to the example of FIG. 4. Also similar to the example of FIG. 4, a gum pocket image 130a′ associated with the contrast-enhanced area 120′ is shown to include an open end and a base end, with the base end being at a location 314 where the tip of a periodontal probe can be positioned during a probing procedure.

Unlike the example of FIG. 4, FIG. 6 shows that in some embodiments, a location 312 of the open end of the gum pocket image 130a′ can be obtained from the contour 302 obtained from the surface image data. For example, the location 312 can be estimated to be where a contour of the tooth surface meets a contour of the scanned surface of the gum (e.g., 12a in FIG. 1). By way of an example, and as depicted in FIG. 6, such a meeting point depicted as 312 can be identified by a significant change in direction (e.g., a sharp bend) in the contour 302 at or near a corresponding gumline. Thus, a length measurement (depicted as length of an arrow 316) between the open end location 312 and the base end location 314 of the gum pocket image 130a′ can provide a gum pocket measurement without a physical probing procedure.

It is noted that in the example of FIG. 6, the contour 302 of the gum tissue (obtained from the surface image data) is depicted as being substantially aligned with the boundary 202 of the gum tissue obtained from the CT image with the contrast-enhanced area 120′ (obtained with the presence of the fluid 120 (FIG. 2). However, and as described herein in reference to FIG. 4, it will be understood that the boundary 202 of the gum tissue may be deviate from the contour 302 of the gum tissue due to gum tissue variations that may be manifested when the gum tissue is subject to pressure associated with application of a fluid such as the image-enhancing fluid (120 in FIG. 2).

Accordingly, it is further noted that in the example of FIG. 6, the open end location 313 of the gum pocket image 130′ can be obtained independent of any gum tissue variations described herein in reference to FIG. 4.

In the various examples of FIGS. 1 to 6, and referring more particularly to FIGS. 4 and 6, the boundary 204 provided by the inner surface 204 of the periodontal device and the boundary 202 of the corresponding gum tissue can result in the contrast-enhanced area 120′ including a first branch-like structure on one side of the gum tissue (e.g., on the left side corresponding to the gum pocket image 130′) and a second branch-like structure on the other side of the gum tissue (on the right side corresponding to the space between 202 and 204). In some applications, the presence of such a second branch-like structure may be problematic during, for example, identification of a gum pocket image structure.

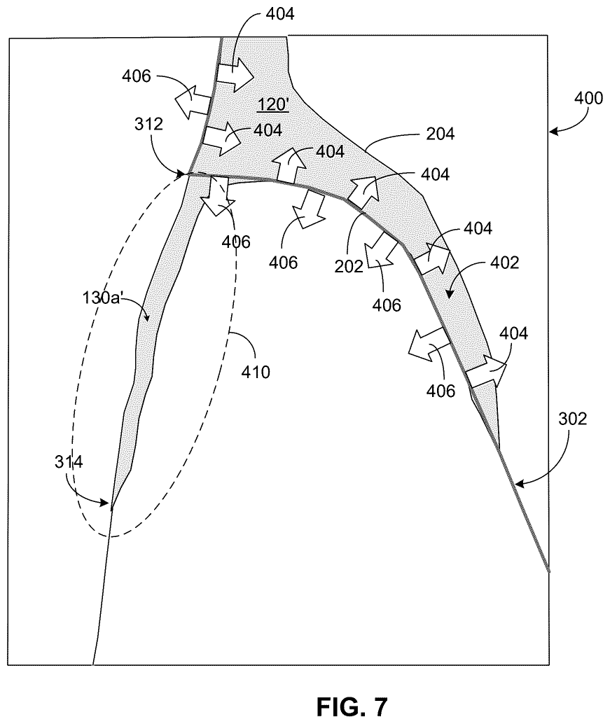

In some embodiments, the foregoing ambiguity associated with a second branch-like structure in CT images can be eliminated or reduced. For example, FIG. 7 show that in some embodiments, ambiguity can be removed or reduced during an analysis of a CT image such as the combined-data image 300 of FIG. 5.

In FIG. 7, an enlarged view of an area 400 (which is similar to the enlarged area 310 of FIG. 6) shows that with use of the contour 302 obtained from the surface image data, the combined-data image can be separated into first and second regions. For example, a first region including the gum pocket image 130′ can be selected to be on the interior side of the contour 302, as depicted by arrows 406, and a second region including the second branch-like structure 402 can be selected to be on the exterior side of the contour 302, as depicted by arrows 404. Once such first and second regions (406, 404) are identified, the second region 404 can be excluded, thus excluding the second branch-like structure 402, during an analysis of the gum pocket image 130′.

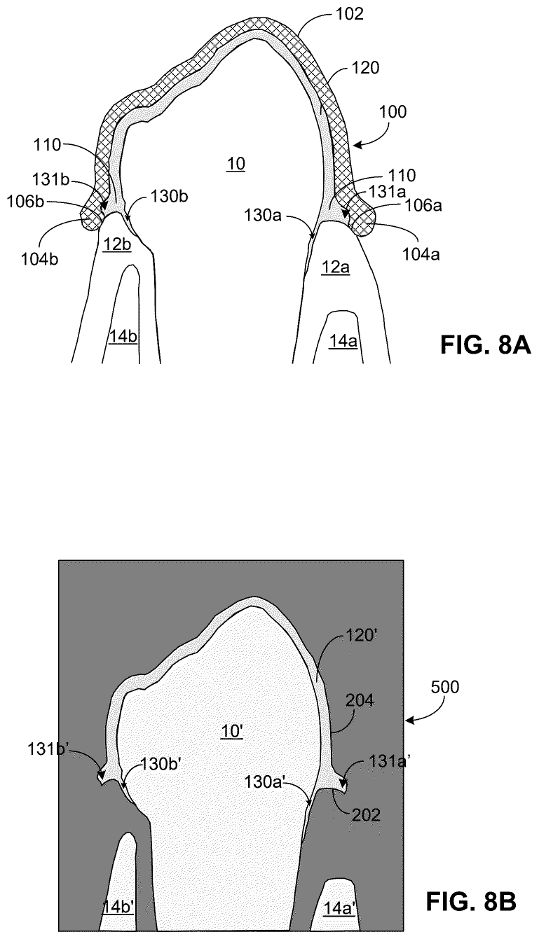

In another example, FIGS. 8A and 8B shows that in some embodiments, ambiguity can be removed or reduced by configuring the periodontal device itself. More particularly, FIG. 8A shows a sectional view of a periodontal device 100 with a space 110 occupied with a fluid 120 similar to the example of FIG. 2, and FIG. 8B depicts a CT image 500 representative of the in-use example of FIG. 8A.

However, unlike the periodontal device 100 of FIG. 2, FIG. 8A shows that in some embodiments, the periodontal device 100 (of FIG. 8A) can be configured such that an extension of a second branch-like structure (e.g., 402 in FIG. 7) in the corresponding CT image 500 is reduced or eliminated sufficiently to reduce or eliminate ambiguity with respect to the gum pocket image 130′ during analysis. For example, the periodontal device 100 of FIG. 8A is shown to be configured such that its end portions 104a, 104b are dimensioned to be closer to the respective gumlines so that respective fluid-occupied spaces 131a, 131b are closer to the gumlines. Accordingly, the resulting structures 131a′, 131b′ in the CT image 500 are shown to have the reduced or no extensions as second branch-like structures.

In some embodiments, the end portions 104a, 104b of the periodontal device 100 of FIG. 8A can be configured to provide sufficient sealing properties when worn by a user. For example, even though the end portions 104a, 104b are closer to the respective gumlines than the example periodontal device 100 of FIG. 2, sealing portions 106a, 106b can be configured to provide sufficient sealing functionality by engagement with the corresponding gum tissue surface, with pressure provided by the user biting down on the periodontal device 100, or some combination thereof.

In some embodiments, a periodontal device (e.g., 100 in FIG. 2) having one or more features as described herein can be configured to be used as follows. For example, such a periodontal device can be implemented as a tray tailored to fit an upper and/or lower arch of a given patient. Such a tray can be provided with a radiocontrast agent in an appropriate form, where such an agent can be pre-packaged in the tray, provided just prior to use, or some combination thereof.

In some embodiments, such an agent can be selected to not drip away prior to application onto the patient and provide a fluid with an appropriate viscosity to allow the radiocontrast agent to enter various gum pockets. For example, the prepared tray containing the radiocontrast agent can be fit over the corresponding arch(es), such that the fitting action includes the corresponding teeth to move into corresponding spaces of the tray thereby providing a pressurized environment for the radiocontrast agent now confined due to the sealing portions (e.g., 106 in FIG. 2) of the tray. Such a pressure can be utilized to force the fluid containing the radiocontrast agent into the gum pockets.

In some embodiments, an antiseptic agent and/or a propulsion agent can be provided in the foregoing tray. For example, a peroxide-based material can be provided in an appropriate form, and such a material can provide both antiseptic functionality and additional pressure for forcing of the radiocontrast agent into the gum pockets.

In some embodiments, a treatment agent can be provided in the foregoing tray, with or without the antiseptic and/or propulsion agents. For example, such a treatment agent can be selected to be forced into the gum pockets along with the radiocontrast agent to thereby provide periodontal treatment as well as periodontal imaging as described herein.

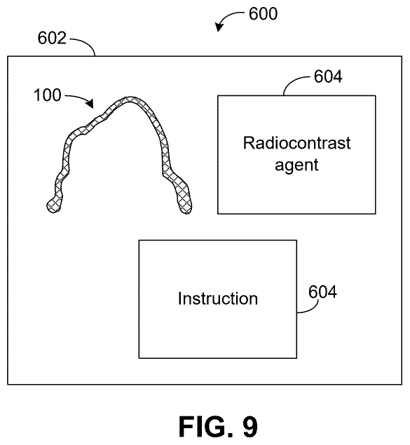

FIG. 9 shows that in some embodiments, a periodontal device 100 having one or more features as described herein can be part of a kit 600. Such a kit can include the periodontal device 100 such as a tray provided within a packaging 602 suitable for periodontal applications. In some embodiments, the kit 600 can further include a radiocontrast agent 604 that can be used with the periodontal device 100 as described herein. In some embodiments, the kit 600 can further include an instruction or information 604 concerning use of some or all parts of the kit 600.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Some aspects of the systems and methods described herein can advantageously be implemented using, for example, computer software, hardware, firmware, or any combination of computer software, hardware, and firmware. Computer software can comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that can be implemented using software to be executed on a general purpose computer can also be implemented using a different combination of hardware, software, or firmware. For example, such a module can be implemented completely in hardware using a combination of integrated circuits. Alternatively or additionally, such a feature or function can be implemented completely or partially using specialized computers designed to perform the particular functions described herein rather than by general purpose computers.

Multiple distributed computing devices can be substituted for any one computing device described herein. In such distributed embodiments, the functions of the one computing device are distributed (e.g., over a network) such that some functions are performed on each of the distributed computing devices.

Some embodiments may be described with reference to equations, algorithms, and/or flowchart illustrations. These methods may be implemented using computer program instructions executable on one or more computers. These methods may also be implemented as computer program products either separately, or as a component of an apparatus or system. In this regard, each equation, algorithm, block, or step of a flowchart, and combinations thereof, may be implemented by hardware, firmware, and/or software including one or more computer program instructions embodied in computer-readable program code logic. As will be appreciated, any such computer program instructions may be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, may be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that can direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or modules stored in a memory or other non-transitory computer-readable storage medium or device. The various functions disclosed herein may be embodied in such program instructions, although some or all of the disclosed functions may alternatively be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1. A method for measuring a periodontal condition, the method comprising:
providing a radiocontrast agent to a region about a tooth and a respective gum tissue, such that some of the radiocontrast agent enters a gum pocket between the tooth and the gum tissue;
obtaining radiograph data representative of an image that includes a pocket image resulting from the presence of the radiocontrast agent within the gum pocket; and
determining a depth dimension of the gum pocket from the pocket image.

2. The method of claim 1, wherein the radiocontrast agent includes a contrast-enhancing material in a fluid form.

3. The method of claim 1, wherein the determining of the depth dimension includes estimating locations of open and base ends of the pocket image.

4. The method of claim 3, wherein the estimating of the locations of open and base ends of the pocket image is based on the image.

5. The method of claim 4, further comprising obtaining surface data for the tooth and the gum tissue, such that the estimating of the location of the open end of the pocket image is based on the surface data.

6. The method of claim 5, wherein the radiograph data includes computed tomography (CT) radiograph data.

7. The method of claim 6, wherein the CT radiograph data includes cone beam computed tomography (CBCT) radiograph data.

8. The method of claim 5, wherein the surface data includes scan data.

9. The method of claim 8, wherein the scan data includes intraoral scan data.

10. The method of claim 1, wherein the providing of the radiocontrast agent includes providing a fluid that includes the radiocontrast agent, such that the fluid is forced into the gum pocket.

11. The method of claim 10, wherein the fluid further includes a propulsion agent to enhance the forcing of the fluid into the gum pocket.

12. The method of claim 10, wherein the fluid further includes an antiseptic agent.

13. The method of claim 10, wherein the fluid further includes a treatment agent selected to provide treatment within the gum pocket.

14. The method of claim 1, wherein the determining of the depth dimension of the gum pocket includes identifying the pocket image on one side of the gum tissue and another region image on the other side of the gum tissue, such that the other region image is excluded in the determining of the depth dimension of the gum pocket.

15. The method of claim 14, wherein the excluding of the other region image is based on a dimension of the other region image.

16. The method of claim 14, wherein the excluding of the other region image is based on a surface data including a scan of the tooth and the respective gum tissue, such that the other region image is selected to be exterior to a surface of the gum tissue.

17. A periodontal device comprising a tray for a patient, the tray configured to provide a radiocontrast agent to a region about a tooth of the patient and a respective gum tissue, such that some of the radiocontrast agent enters a gum pocket between the tooth and the gum tissue when the tray is worn by the patient, such that a radiograph image of the tooth and the gum tissue includes a pocket image resulting from the presence of the radiocontrast agent within the gum pocket thereby allowing determination of a depth dimension of the gum pocket from the pocket image.

18. The periodontal device of claim 17, wherein the tray is configured to fit over an arch of the patient, such that the tooth is one of a plurality of teeth of the arch.

19. The periodontal device of claim 17, wherein the tray is configured to allow determination of the depth dimension of the gum pocket by identification the pocket image on one side of the gum tissue and another region image on the other side of the gum tissue, such that the other region image is excluded in the determining of the depth dimension of the gum pocket.

20. The periodontal device of claim 19, wherein the tray is configured such that its end on the one side of the gum tissue is dimensioned differently from its end on the other side of the gum tissue, thereby allowing the exclusion of the other region image based on a dimension of the other region image.

21. A system for measuring a periodontal condition, the system comprising:
a radiocontrast application component configured to deliver a radiocontrast agent to a region about a tooth and a respective gum tissue, such that some of the radiocontrast agent enters a gum pocket between the tooth and the gum tissue;
a radiograph component configured to obtain radiograph data representative of an image that includes a pocket image resulting from the presence of the radiocontrast agent within the gum pocket; and
a computing component configured to determine a depth dimension of the gum pocket from the pocket image.

22. A periodontal kit comprising:
a packaging suitable for periodontal application;
a tray for a patient, the tray configured to provide a radiocontrast agent to a region about a tooth of the patient and a respective gum tissue, such that some of the radiocontrast agent enters a gum pocket between the tooth and the gum tissue when the tray is worn by the patient, such that a radiograph image of the tooth and the gum tissue includes a pocket image resulting from the presence of the radiocontrast agent within the gum pocket thereby allowing determination of a depth dimension of the gum pocket from the pocket image; and
an instruction for use of the tray.

23. The periodontal kit of claim 22, further comprising the radiocontrast agent.
